(19) United States
(12) Patent Application Publication
(10) Pub. No.: US 2004/0106158 A1
(43) Pub. Date: Jun. 3, 2004

(54) IP3 protein binding assay

(76) Inventors: Tabassum Naqvi, Fremont, CA (US); Riaz Rouhani, Concord, CA (US); Peter A. Fung, Sunnyvale, CA (US); Richard M. Eglen, Los Altos, CA (US); Rajendra Singh, San Jose, CA (US)

Correspondence Address: Hana Verny, Peters, Verny, Jones & Schmitt LLP, Suite 230, 425 Sherman Avenue, Palo Alto, CA, 94306, US

(21) Appl. No.: 10/689,122

(22) Filed: Oct. 20, 2003

Related U.S. Application Data
(60) Provisional application No. 60/420,469, filed on Oct. 21, 2002.

(51) Int. Cl. G01N 33/53

(52) U.S. Cl. 435/7.9

(57) ABSTRACT

Protein binding assays are provided for determining IP₃ in a sample employing as reagents a conjugate of IP₃ joined at the 2-oxy through a bond or linking group to a detectable label and a truncated portion of the extracellular fragment of an IP₃R. The reagents are combined with the sample and the amount of IP₃ determined by means of the detectable label. The conjugate with the enzyme donor fragment of β-galactosidase or a fluorescer is specifically described.

### RELATED APPLICATIONS

This application claims priority of U.S. provisional patent application No. 60/420,469, which is specifically incorporated herein by reference.

### BACKGROUND OF THE INVENTION

1. Field of the Invention

The invention concerns the measurement of IP₃ (D-myo-inositol,1,4,5 trisphosphate).

2. Background Information

IP₃ plays an essential role as a second messenger regulating cellular Ca⁺⁺ by controlling the release of calcium from calcium stores in the endoplasmic reticulum into the cytoplasm. The receptor for IP₃ is a gated calcium release channel residing at the calcium storage sites. IP₃ is one of a family of phosphorylated inositol compounds that play different roles. The inositol family of phosphate esters differ as to the number of phosphates, the position of the phosphates, as well as their stereochemistry, so as to include both geometric and stereochemical isomers. A family of phosphatases and kinases provide for rapid interchange between the different inositol phosphates. Because of the importance of calcium levels in the cytoplasm, IP₃ is an analyte of great interest.

Measurement of intracellular second messengers such as cAMP or IP₃ has typically been used to decipher signaling events in the cell mediated through GPCRs. GPCRs constitute the largest subgroup (about 45%) of all the molecular targets that are currently being pursued in drug discovery programs. These receptors transduce the binding of extracellular ligands into intracellular signaling events that are mediated through guanine nucleotide binding regulatory proteins (G-proteins). Traditionally, drug discovery programs targeting GPCRs have relied on the use of tissue preparations from native sources to perform screens of medicinal and natural product libraries.

The number of similar inositol phosphates makes IP₃ a difficult target to analyze. Also, the simplicity of the molecule and its low antigenicity makes it difficult to generate high affinity antibodies to IP₃. Any modification of IP₃ changes the character of the molecule, so that in any competitive assay where a derivative must be used, the modification must not significantly change the affinity of the labeled derivative as compared to the naturally occurring IP₃. For the most part the assays for IP₃ have depended upon using radioactive tags where the labeled compound is chemically identical to the naturally occurring IP₃.

While radioactive isotopic assays have high sensitivity and provide a labeled analog that can successfully compete for proteins binding IP₃, there are many undesirable aspects about using radioactive isotopes as a label. The use of radioactivity is dangerous, has serious disposal problems and since the time of Berson and Yalow's discovery of radioimmunoassay, the diagnostic field has moved away from the use of radioactive labels, to such other labels as fluorescers, enzymes, particles, enzyme fragment complementation and the like. There is a substantial interest in developing assays that avoid the use of radioisotopes, while providing the necessary sensitivity and specificity for detecting IP₃, without interference from the other inositol phosphate congeners.

### DESCRIPTION OF RELEVANT LITERATURE

Derivatives of IP₃ are described in Marecek, et al., Carbohydrate Res. 1992, 234, 65-73; Guo, et al., Bioorg & Biochem. 1994, 2, 7-13; Liu and Potter, J. Org. Chem. 1997, 62, 8335-40; and Chen, et al., J. Org. Chem. 1996, 61, 393-7. Methods for analytical separation of inositol phosphates are illustrated in U.S. Pat. No. 5,225,349 and Hamada, J. Chromatog. A, 2002, 944, 241-8. Radioactive protein binding assays are described in Anderson, et al., J. Chromatog. 1992, 574, 150-5; Hingorani and Agnew, Anal. Biochem. 1991, 194, 204-13; and Bredt, et al., Biochem. Biophys. Res. Commun. 1989, 159, 976-82. Antibodies for IP₃ and the derivatives used for preparing the antibodies are described in Shieh and Chen, Biochem. J. 1995, 311, 1009-14; Chen and Chen, et al., U.S. Pat. Nos. 5,393,912 and 5,798,447 and PCT application serial no. WO95/19373. Binding proteins other than antibodies for IP₃ are described in U.S. Pat. No. 6,087,483; EPA 0,992,587 and Uchimaya, et al., J. Biol. Chem. 2002, 277, 8106-113. U.S. Pat. No. 5,252,707 describes the preparation of derivatives of IP₃. Packard Bioscience, Alpha Screen Technology, Application Note ASC-018, Homogeneous Inositol 1,4,5-Trisphosphate (IP₃) Functional Assay for the G_{q}-coupled AT1 Receptor, describes a homogeneous assay for IP₃. The use of fluorescence polarization in assays is described by Owicki, “Fluorescence Polarization and Anisotropy in High Throughput Screening: Perspectives and Primer”, Journal of Biomolecular Screening 2000, 5, 297-306.

The presence of thiol groups in IP₃R is described in Kaplin, et al., 1994 J Biol Chem 269, 28972-78.

References describing the use of pleckstrin homology (PH) proteins for binding to vicinal diphosphate inositols include Hamman, et al., J. Biomol. Screening 2002, 7, 45-55; Dowler, et al., Biochem. J. 2000, 351, 19-31; and Lemmon and Ferguson, 2000, Biochem. J. 2000, 350 1-18. A fluorescent in vitro biosensor for IP₃ using a fluorophore labeled pleckstrin homology domain has been described by Morii et al., J. Am. Chem. Soc. 2002, 124, 1138-9.

### SUMMARY OF THE INVENTION

Sensitive and specific non-radioactive protein binding assays are provided using an IP₃ derivative labeled at the 2-position and a truncated IP₃ receptor protein. The label is a small molecule of less than about 10 kD. The sample is processed to inactivate phosphatases and kinases, combined with the above-indicated reagents and the amount of bound or unbound label determined. Particularly enzyme fragment complementation and fluorescent polarization are used for detection.

### BRIEF DESCRIPTION OF THE FIGURES

FIG. 1 is a calibration curve of an assay using enzyme fragment complementation to determine IP₃ concentration;

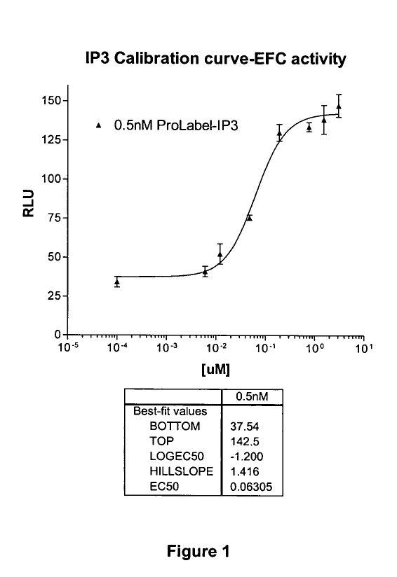

FIG. 2 is a graph of the effect of the addition of DTT to the binding protein buffer on stability of the assay;

FIG. 3a is a fluorescence polarization calibration curve using IP₃ binding protein in PD 10 buffer in the presence and absence of DTT;

FIG. 3b shows the of the results of ligand induced IP₃ production from CHO-M1 cells measured by fluorescence polarization.

FIG. 4 is a table of results and a graph of the results using Cy3B fluorescer in an IP₃ fluorescence polarization assay;

FIG. 5 is a table of results and a graph of the results using hexachlorofluorescein fluorescer in an IP₃ fluorescence polarization assay;

FIG. 6 is a table of results using Alexa fluorescer in an IP₃ fluorescence polarization assay;

FIG. 7 is a graph of the results using fluorescence polarization and carbachol induction on an ATCC CHO-M1 cell line; and

FIG. 8 is a bar graph of the determination of IP₃ at basal level with three different cell lines counting the number of cells.

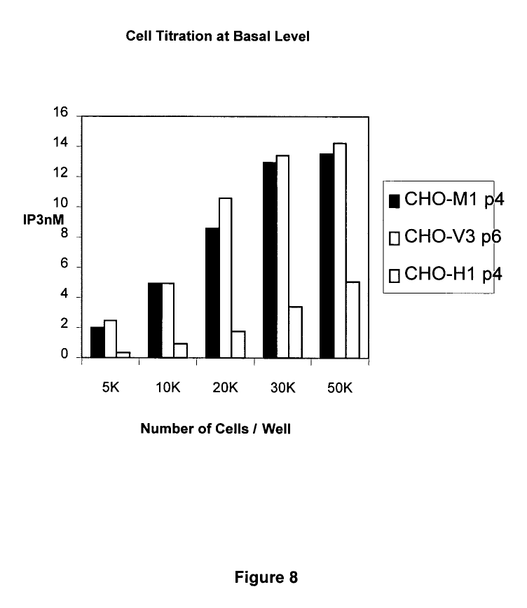

### DESCRIPTION OF THE SPECIFIC EMBODIMENTS

In accordance with the subject invention non-radioactive protein-binding assays for IP₃ are provided. Cellular samples are processed to inactivate kinases and phosphatases to retain the naturally occurring IP₃ concentration. The sample may be further processed or modified, either before or after inactivation to prepare the sample for the assay. The processed sample is combined with a labeled IP₃, where the label is a derivative joined at the 2-position, particularly through an ether or ester group, usually through a linker. Depending upon the nature of the label, the molecular weight range will vary. For fluorescent labels, the label will usually be under 2 kD, more usually under 1 kD, while for the enzyme label, the label will be less than about 30 kD, usually less than about 10 kD, preferably less than about 8 kD. Therefore, the label will generally range in molecular weight from about 0.2 kD to up to about 30 kD.

A high affinity binding protein derived from an IP₃ receptor is employed as the binding protein. The sample and reagents are combined where the labeled derivative competes with the sample IP₃ for binding to the binding protein. Either or both the bound or unbound label may be determined. Of particular interest are homogeneous protein binding assays, which avoid a separation step after the combining of the sample and reagents.

In describing the invention, the reagents employed will be considered first. The labeled derivative or IP₃ analog will have a hydrogen bonded to oxygen at the IP₃ 2-position replaced, directly or usually through a linker to a detectable label, where the detectable label can provide a signal directly or indirectly, that is, additional reagents may be required. The linker includes the functionality, if any, to which the label is bonded and any functionality bonded to the 2-hydroxyl. The remaining portion of the linker other than the terminal functional groups will be referred to as the linking group, which may be a bond, but will usually be a chain. The chain will usually be of at least 2 atoms with a carbon atom bonded to the oxygen at the IP₃ 2-position, there being not more than about 16 atoms in the chain, usually not more than about 12 atoms in the chain (for a cyclic group the shortest link will be counted), where the atoms are carbon, nitrogen, oxygen, sulfur and phosphorous, where carbon atoms and heteroatoms may be in the chain or as substituents bonded to atoms in the chain. The linker will usually be at least one atom other than hydrogen and not more than about 30 atoms other than hydrogen, usually in the range of about 4 to 25 atoms. For the most part, the linker will be neutral or anionic, although cationic groups may be present, but will usually not be employed. The functional groups that are employed for the linker and the attaching functionality will be described below. The attaching functionality for the label will vary widely depending upon the nature of the label, where synthetic convenience, absence of interference with the assay, and high affinity of the derivative will direct the functionality that is employed.

The labeled derivative composition may not be a pure composition, generally having at least about 75% of the 2-position derivative, particularly at least about 90% and more particularly approaching at least 99%, preferably 100%, of the 2-position derivative. The cations are not considered as part of the derivative composition, since they will ionize in solution. For the most part the cations will be ammonium and alkali metal cations.

The labeled derivative will for the most part have the following formula:

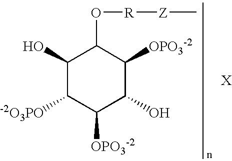

wherein:

R is a bond or linking group, usually a linking group of at least about 1 atom, usually at least about 2 atoms and more usually at least about 4 atoms, other than hydrogen, wherein said atoms include at least one carbon atom, there being not more than about 16 atoms in the chain, usually not more than about 12 atoms in the chain, which besides carbon atoms, may include the heteroatoms nitrogen, phosphorous, oxygen and sulfur, there generally being from 0 to 6, more usually 0 to 4 heteroatoms, more usually 1 to 4 heteroatoms, wherein the linking group may also include such heteroatoms as substituents on the chain, including oxo, amino, oxy, and thio. R may be aliphatic, alicycyclic, aromatic or heterocyclic, or combinations thereof, particularly aliphatic, branched chain or straight chain, saturated or unsaturated, having not more than about 2 sites of unsaturation, usually saturated. Usually, the linking group will be neutral or negatively charged, preferably neutral, having from 1 to 3, usually 1 to 2 heterogroups. The linking group may be hydrophilic or hydrophobic.

Z is a functionality bonded to R linking the label to R and may include oxy, amido, thio, succinimidyl, amino, ureido, ester, phospho, thiophospho, oxalo, etc., or combinations thereof, generally being of a total of from about 1 to 10 atoms, including carbon atoms and heteroatoms. Any functionality may be used for linking that does not interfere with the role of the reagent, the group being chosen because of synthetic convenience, stability and lack of detrimental effect;

X is the label, generally of from about 150 Dal to about 30 kD or optionally higher, usually not more than about 10 kD, preferably not more than about 6 kD, except when a surface or insoluble label, where the molecular weight may be indeterminate. The label may be varied widely being selected to provide the desired sensitivity for the assay, the absence of interference from the other reagents in the assay, the absence of interference of binding of the derivative to the binding protein, and having a reasonable protocol, generally avoiding a separation step after the combining of the sample with the reagents; and

n is an integer of from 1 to 2 depending upon the nature of the label, usually being 1 with a fluorescent label and 1 or 2 with an enzyme donor (“ED”) label (to be subsequently described).

The group bonded to the 2-hydroxyl may be a saturated carbon atom or carbonyl, including thiocarbonyl, usually oxo-carbonyl. Depending upon the nature of the label, one or more IP₃'s may be bound to the label.

Any label that provides a signal sufficiently sensitive to detect the dynamic range of IP₃ without any interference with the assay can be employed. There is an enormous diversity of labels that may find use. Labels that have found use in assays include enzymes, e.g. G6PDH, malate dehydrogenase, horseradish peroxidase, β-galactosidase, etc., which enzymes are not preferred due to their high molecular weight; enzyme fragments in complementation assays, e.g. EDs from β-galactosidase, β-lactamase, ribonuclease, e.g., ribonuclease S, etc.; fluorophores, that can be detected by, for example, fluorescence, fluorescence polarization, time resolved fluorescence or fluorescence correlation spectroscopy; gold sol particles which upon aggregation change color; enzyme cofactors such as FAD or heme that complex with an apoenzyme when not sterically blocked by binding to a receptor; enzyme inhibitors, such as ethoxymethylphosphonothioates for acetylcholinesterase and methotrexate for DHFR, that complex with an enzyme when not sterically blocked by binding to a receptor; electroactive labels, such as ferrocene and Ru(II) chelates, which can be detected upon binding to a receptor at an electrode; latex; chemiluminescent labels; and the like. These assays may be found in E F Ullman, pp 177-94. The Immunoassay Handbook 2^{nd} Ed, David Wild ed, Nature Publishing Group 2001, as well as in numerous publications, Letters Patent and product inserts. In addition, other assays that can find use include mass tags, detectable by mass spectrometry or electrophoresis, metal chelates detectable by flame ionization, oligonucleotides detectable by amplification, e.g. PCR.

Labels of particular interest include enzyme complementation fragments, such as the enzyme donor (ED) fragment from β-galactosidase and β-lactamase, fluorescers, and chemiluminescers, particularly the enzyme complementation fragments and fluorescers. (The ED is commonly referred to in the literature as an enzyme donor, being the smaller fragment as compared to the EA, the enzyme acceptor.) One can achieve complementation by either having an ED labeled ligand complex with EA, or make fusion proteins of the ED and EA, with complementary binding agents that will naturally complex and bring the ED and EA together. Of particular interest is the use of fragment complementation enzyme donors as employed with a small ED fragment of β-galactosidase (the small ED is also referred to as Prolabel or PL). The enzyme donor will be at least about 36 amino acids and not more than about 95 amino acids, usually not more than about 75 amino acids. It is found that a relatively large polypeptide does not interfere with the binding of the IP₃ derivative to the binding protein.

For ED, the ED may have one or two functionalities for linking. Of particular interest is where the ED has one to two thiol groups, generally as cysteines proximal to or at the termini of the ED, which thiol groups may be added to an activated olefin to form a thioether.

Fluorescent assays are also of particular interest. The equation for fluorescence polarization is mP=(F⊥−F_{[}/F⊥+F_{∥})×1000. Fluorescers of interest include fluorescein, rhodamine, umbelliferone, the squaraines, the cyanine dyes, e.g. Cy3, Cy5, Cy5.5, etc., (available from Amersham Biosciences), Bodipy, AlexaFluor (available from Molecular Probes), and time resolved fluorescers, such as chelates of the actinides and lanthanides, e.g. Th, Eu, Er, Sm, Yt, etc. See, for example, U.S. Patent no. 6,455,851. Several fluorescence detection methods can be used for binding assays. These include the measurement of the fluorescence of either the bound or unbound label following separation of these components. Time resolved detection of fluorescence is particularly useful in this application because it helps discriminate weak signals from background fluorescence. Homogeneous methods include fluorescence correlation spectroscopy in which the rate of diffusion of individual molecules provides information on the fraction of bound and unbound label, FRET assays in which energy is transferred between two different dyes in the bound complex when one is attached to the receptor and one to the tracer, and fluorescence polarization in which measurement of the change in the polarization of the emitted light is associated with binding of the tracer to the receptor.

Fluorescence polarization assays have found widespread use because of relatively simple instrumentation and the requirement for only a single dye. A key factor in the performance of fluorescence polarization-based assays is the change in polarization upon binding of the tracer to a receptor. Dyes that produce less perturbation of receptor-binding affinity and other activity are preferred. Desirably, the polarizability of the unbound dye should be less than about 0.04 polarization units (p), preferably less than about 0.03 p although dyes having as high as 0.06 p can be used (mP=10⁻³ p). Long-wavelength emitting dyes that tend to minimize assay interferences due to intrinsically fluorescent samples such as cell lysates are preferred. For time resolved fluorescence preferred dyes include those having longlived excited states,—particularly dyes incorporating fluorescent lanthanides and ruthenium and polycyclic hydrocarbons. For FRET assays combinations of dyes are used, where one dye acts to absorb and transfer the light energy and the other dye acts to receive and emit the light energy. These combinations allow relatively short wavelength sources while emitting at relatively long wavelengths to minimize interference from scattered light and other interference, such as candidate compounds from a library of compounds.

Specific fluorescers that have found use in fluorescence polarization assays are: fluorescein, Biochemistry 33, 10379 (1994); J Biomol Screen 5, 77 (2000); Gene, 259, 123 (2000); and Biotechniques 29, 344 (2000); Bodipy, by itself or in combination with another dye, e.g. tetramethylrhodamine or fluorescein; J Biomol Screen 5, 329 (2000); ibid 7, 111 (2002); Anal Biochem 278, 206 (2000); ibid 247, 77 (1997); ibid 243, 1 (1996); and Antimicrob Agents Chemother 43, 1124 (1999); Oregon green 488, Biochemistry 38, 13138 (1999); and tetramethylrhodamine, Biotechniques 29, 34 (2000).

Fluorescence polarization measurements have long been a valuable biophysical research tool for investigating processes such as membrane lipid mobility, myosin reorientation and protein-protein interactions, Jameson and Seifried, Methods, 1999,19, 222-33. Immunoassays which have been developed and used extensively for clinical diagnostics represent the largest group of bioanalytical applications, however recently, the advent of microplate readers equipped with polarizing optics has led to the adoption of fluorescence polarization as a readout mode for high-throughput screening.

Tracers used in fluorescence polarization assays include peptides, drugs and cytokines that are modified by the attachment of the fluorescent dye. Depolarization due to flexibility in the attachment of the dye, perturbs and distorts the polarization. For this reason, it is generally preferable to use reactive dyes without long aliphatic linkers between the fluorophore and the reactive group in the preparation of tracers for fluorescence polarization-based assays.

Illustrative linking groups linked to the 2-hydroxyl oxygen include propylamidobutyl, propylamidophenyl, propyloxypropyl, butylureidohexyl, phenyl, butylureidophenyl, pentyl, propyl phosphate diester, butyryloxypentyl, dibutyl phosphate ester, N-(N′-ethyl-2-propylamido) butyrylamido, hexylthioethyl, hexylthiophenyl, methoxyacetyl, diethyleneoxy, etc.

Of particular interest are conjugates in assays dependent on a binding protein obtained by truncating the extracellular part of a IP₃R, Type 1, 2 or 3, where there is a substantial enhancement of binding to IP₃ over the natural receptor, usually at least about 200-fold enhancement, preferably at least about 500-fold enhancement, and even 1000-fold enhancement or greater.

A binding protein for IP₃ is specifically described in EPA 0 992 587 and Uchiyama, et al., 2002, supra. These references are incorporated herein in their entirety, as if set forth in haec verba herein. Other IP₃ binding proteins may also be used that are derived from IP₃ receptors, following the procedure employed in the cited references. By isolating the IP₃R or expressing only the extracellular portion of the IP₃R and mildly trypsinizing or using another relatively non-specific protease, large fragments of the extracellular portion can be obtained. These can be isolated using labeled IP₃, e.g. with a radioisotope or biotin (including biotin mimic), employing chromatography, panning, streptavidin bound to a surface, etc. The affinity for IP₃ may then be determined in accordance with conventional assays or according to this invention. Alternatively, one may use labeled core protein employed in the subject invention in competition with truncated fragments of the IP₃R for labeled IP₃ and determine the extent to which the labeled IP₃ binds to the core protein in the presence of the truncated IP₃R fragments. One may then isolate the gene for the IP₃R and by manipulation of the gene determine the minimum number of amino acids of the fragment that maximize the affinity. Methods for identifying such monomer sequence are amply described in the literature including the references cited herein.

The significant factor is that the core protein or “sponge” is readily available and for the purposes of this invention only one protein is required that has the requisite characteristics. The core protein is derived from mouse type 1 IP₃R1. The core protein is amino acids 226-578, although the naturally occurring N- and C-amino acids may be included, usually to provide a protein of not more than 1.5 k amino acids, preferably not more than about 750 amino acids and more preferably not more than about 600 amino acids. The extension need not be the naturally occurring amino acids, and may total 1-500 amino acids, usually not more than about 1 to 300 amino acids. The additional amino acids may serve a variety of purposes, such as aiding in the purification of the core protein, aiding in the isolation of the complex between the core protein and an IP₃ derivative, causing steric inhibition of complementation with a peptide label to its cognate protein, or attachment to a surface or another molecule, where the surface may be a plate, a microtiter well wall, a particle, or the like.

In some instances one may make a fusion protein of the binding protein, where the fused polypeptide may serve a variety of functions, such as ease of purification, enhanced stability under the conditions of the assay, in the use of FRET assays, using GFP and like variants, etc. Generally the fused polypeptide will be less than about 1 kD, usually less than about 0.6 kD and more usually less than about 0.5 kD. Among fusion proteins of the binding protein, a fusion with GST has found use.

The binding protein or sponge has a plurality of thiol groups, namely seven thiol groups. The binding protein is available as a fusion protein with glutathione sulfur transferase, which provides for a total of 11 cysteines. It is found that better results are obtained when including a reductant that inhibits disulfide formation, such as dithiothreitol, bis-imide mercaptoacetyl, mercaptoethylamine, bisulfite, β-mercaptoethanol, etc. The amount of the reductant will generally be in the range of about 1-100 mM.

The IP₃ derivatives can be prepared using the procedures described in U.S. Pat. No. 5, 252,707. Beginning with the 4,5-diphosphate inositol, the 3,4,5 and 6 positions may be selectively protected using an aralkyl halide, e.g. benzyl chloride, leaving the 1- and 2-hydroxyl groups unprotected. The 1-hydroxyl may then be selectively protected using silylation, followed by using the unprotected 2-hydroxyl for nucleophilic substitution on an acyl group or saturated alkyl group having a displaceable functionality, e.g. halide or pseudohalide. The 1-position may then be phosphorylated removing the silyl group and the protecting groups removed providing the 2-derivative of the IP₃.

The assays employed can be homogeneous or heterogeneous protein binding assays, where the analyte IP₃ competes with a labeled IP₃ analog for a binding protein specific for the analyte. In homogeneous assays the binding of the protein to the analog to form a complex results in a change in an observed signal. In heterogeneous assays, the complex of the binding protein and the analog is sequestered to a surface, a well wall, a particle, e.g. magnetic particle, or other surface, where the assay medium may be removed and the bound complex washed, so as to remove any analog from the surface. The presence of the analog on the surface may then be determined. Of particular interest are assays employing enzyme donors in an enzyme fragment complementation assay, more particularly the ED of β-galactosidase, and fluorescers, particularly in fluorescence polarization assays.

For the assay, depending upon whether one is performing an in vitro assay or wishes to do a cellular assay, one may wish to grow cells to partial confluence or confluence for use. Once the cells have been expanded, they may then be harvested for use. Due to the plethora of activities with which IP₃ is involved, a large variety of cells may be employed. The cells may be neuronal, heart, liver, kidney, leukocytes, spleen, skin, muscle, epidermal, endothelial, retinal, mesenchymal, etc. The cells may be naturally occurring, e.g. primary cells, cell lines, genetically modified cells, and may be from any eukaryote, e.g. mammal, such as human, mouse, lagomorpha, porcine, etc.

Depending upon the purpose of the assay, the cells may be subject to prior treatment or used directly. For example, primary cells may be checked to determine their IP₃ content to evaluate the state of the cells. In other situations, one may be interested in the effect of an agent on IP₃ formation, degradation or modification. The agent will usually, but not necessarily, be a drug that is being screened as to its activity, either direct activity on the level of IP₃ or whether the drug has as a side effect an activity affecting the level of IP₃. Where the effect of a change of environment, e.g. presence of a drug, is being determined, the cells will usually be incubated in an appropriate nutrient medium for a period of at least about 5 min and not more than about 6 h. The number of cells required for the assay will usually be in the range of about 10² to 10⁷ more usually in the range of about 10³ to 10⁵. The concentration of IP₃ to be determined will generally be in the range of about 0.1 to 10 nmolar, which is generally about the physiological concentration.

The cells are lysed. Prior to or subsequent to lysing the action of phosphatases or kinases is inhibited to prevent modifications of the inositol phosphates present in the cell. Blocking the enzymatic reaction may be achieved in a variety of ways. Heat may be employed using a pulse of at least about 60-80° C. for a time in the range of about 0.25 to 120 sec followed by rapid cooling. Alternatively, one may use pH, by using a strong acid at a concentration in the range of about 0.1 to 0.25% with the sample, either before or after adding the binding protein and the IP₃ derivative. Acids that find use include perchloric acid, trichloroacetic acid, trifluoroacetic acid, etc. Depending on the nature of the label, it may be necessary to neutralize the acid by using an appropriate base. The sample may be brought to about pH 6.5 to 8. After inactivation, debris and other large components, e.g. organelles, may be removed by centrifugation and the supernatant isolated. Alternatively, the sample may be aspirated.

Sample preparation may follow the procedures described in BIOTRAK cellular communication assays, D-myo-Inositol 1,4,5-trisphosphate (IP₃) [³H] assay system, code TRK 1000, Amersham Pharmacia Biotech. Alternatively, one may follow the procedure described in U.S. Pat. No. 6,183,974, incorporated herein by specific reference, excluding the use of the radioactive myo-inositol. The procedure generally involves seeding cells into wells at a density of about 10⁵-10⁶, culturing for 2 or more days, incubating with a test compound in assay medium at 37° C. at predetermined time periods, and arresting the cellular activity by aspiration and addition of ice-cold 5% TCA. The pH is then adjusted to 7.4 with conc. NaOH and tris base.

The volume of the sample per 100 μl will generally be in the range of about 5 to 50 μl while the concentration of the other reagents will depend upon the nature of the label and will follow the methodology for a particular label.

As illustrative of a particular protocol using the ED of β-galactosidase, the analog is the ED of β-galactosidase linked at the 2-hydroxyl of IP₃. Usually, the ED will have from 37 amino acids to about 90 amino acids, more usually up to about 60 amino acids, preferably not greater than about 56 amino acids. The binding protein is a truncated extracellular portion of an IP₃R, particularly the core protein described by Uchiyama, 2002, supra, from mouse IP₃R1, including amino acids 200 to 610, more particularly 226 to 578. After blocking the phosphate related enzymes, and modifying the sample as appropriate, e.g centrifugation, a volume of 5-50 μl of the sample is combined with a volume of about 5-50 μl of the binding protein to provide a total concentration in the final assay mixture in the range of about 0.1 nM to 1 μM more usually in the range of about 1 to 100 nM. The mixture is then incubated, conveniently at room temperature for at least 1 min, usually at least about 5 min and not more than about 30 min, there being no advantage in unduly extending the incubation period. At the end of the first incubation, about 5-50 μl of the analog is added with the ED joined to the IP₃ at the 2-hydroxyl by a linking group including the attaching functionalities, generally of from 4 to 20 carbon atoms, where the final concentration of the analog in the assay medium will usually be in the range of about 10 pM to 100 nM, more usually in the range of about 0.1 nM to 10 nM. The mixture is then incubated for the period as described above for the first incubation.

After the second incubation EA is added in a volume of about 5 to 50 μl and the mixture incubated for at least about 5 min, usually at least about 10 min and not more than about 60 min, usually not more than about 45 min. Generally the amount of EA will be at least equal to the concentration of the ED, usually in excess, generally not more than about 10-fold excess, more usually not more than about 5-fold excess. At this time about 5 to 50 μl of a substrate providing a detectable signal is added, where the substrate is in substantial excess of the amount that will be turned over in the assay. Illustrative substrates, many of which are commercially available, include dyes and fluorescers, such as X-gal, CPRG, 4-methylumbelliferyl β-galactoside, resorufin β-galactoside, Galacton Star (Tropix, Applied Biosystems). The procedure follows the conventional procedure for other analytes described in the scientific and patent literature. See, for example, U.S. Pat. Nos. 4,708,929 and 5,120,653, as illustrative. The assay mixture may then be read at a specific time, e.g. 1-10 min, or as a rate, taking readings at specific intervals. With a chemiluminescent readout, the signal may be integrated for a time period of from 0.1 s to 1 min.

To further enhance sensitivity, one may add antibodies to the binding protein to further enhance the bulk around the ED. Antibodies can be antisera or monoclonal, preferably monoclonal. The antibodies would be added after incubation with the binding protein and the sample and IP₃ analog. The antibodies will generally be in a mole ratio of at least about 1:1 to the core protein and generally at least about 2:1, where there may be used antisera that binds to a plurality of epitopic sites on the core protein or one or more monoclonal antibodies where the different antibodies bind to different sites on the binding protein. The incubation with the antibodies can be in the time range of the other incubations.

Another protocol of specific interest is fluorescence polarization, where the label is a fluorescer. The methodology is well established and has been described in numerous patents, including devices for measurement, such as U.S. Pat. Nos. 6,455,861; 6,159,750; and 4,952,691. In performing the method, a sample suspected of containing IP₃ is mixed with the binding protein. By maintaining the concentration of the analog and binding protein and the ratio of analog and binding protein, the ratio of IP₃ complex to analog complex is directly proportional to the amount of IP₃ in the sample. Upon exciting the assay mixture with fluorescent light, particularly at or about the absorption maximum of the fluorescer, and measuring the polarization fluorescence emitted by the fluorescer, one is able to quantitatively determine the amount of IP₃ in the sample. The assay may be performed in any convenient buffer, e.g. borate, phosphate, tris, etc., at a temperature in the range of about 15 to 40° C., using some of the principles described above, such as order of addition and incubation.

The indicated specific assays have many advantages. They are highly specific, not subject to interference from other inositol phosphates, rapid, can be automated and have high sensitivity. As shown in the experimental section, the ED assay has a dynamic range of from 1.0 to 10³ nM. Sensitivity increases with diminishing concentration of IP₃.

For convenience, the reagents can be provided in kits. Depending upon the specific label different components may be included. Each of the kits will include the IP₃ conjugate, and the binding protein as described previously, and may include instructions for performing the assay, particularly electronically encoded or written instructions, buffer, etc. For the enzyme fragment conjugate, there will also be included the enzyme acceptor fragment and substrate for the holoenzyme. Also included is a reductant, conveniently a thiol, more particularly a polythiol, such as dithiothreitol.

### EXPERIMENTAL

### Example 1

A. Preparation of the D-myo-inositol-2-O-(2-(3-maleimidopropionyl)aminoethyl)-4,5-triphosphate(mp-2-O-ae-1,4,5-IP3).

D-myo-inositol-2-O-(2-aminoethyl)-1,4,5-triphosphate(2-O-ae-1,4,5-IP₃) was prepared according to a published procedure. Riley and Potter, Chem Commun, 2000, 983-984. To a solution of 2-O-ae-1,4,5-IP3 (1 mg) in sodium phosphate (100 mM, pH 8.0, 1 mL) was added 100 μL of dry acetonitrile. Succinimidyl-3-maleimidopropionate (3 mg) was dissolved in minimum of acetonitrile (˜200 μL). The maleimide solution was slowly added to the amine solution and the reactants mixed by vortexing. The mixture was allowed to stand for 10 minutes. The product was isolated by HPLC and identified by FAB mass spectroscopy.

B. Preparation of the PL47mdiCys conjugate of D-myo-inositol-1-(3-(3-maleimidopropionyl) aminopropyloxy)-4,5-triphosphate (PL47m-(mp-1P-ap-1,4,5-IP3)₂).

To a solution of freshly desalted PL47mdiCys (˜0.5 mg, 93 nmoles) in sodium phosphate (100 mM, pH 7.0) was added mp-2-O-ae-1,4,5-IP3 (0.35 mg, 557 nmoles) in water. (PL47mdiCys is amino acids 4 to 51 of E. coli β-galactosidase with cysteines added at both termini.) The mixture was reacted for 60 min. The product was purified by preparative HPLC using a gradient of 100 mM triethyl ammonium acetate (pH 7.0) and acetonitrile. Fractions containing the conjugate were identified by MALDI-TOF mass spectroscopy.

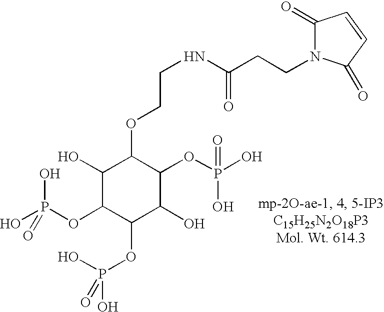

Preparation of the 2-O-(2-aminoethyl-(6-carboxamidofluoresceinyl))-D-myo-inositol-1,4,5-triphosphoric acid.

2-O-(2-aminoethyl)-D-myo-inositol-1,4,5-triphosphoric acid is prepared by the method of Riley and Potter, Chem. Comm., 983-984, 2000. To a solution of 2-O-(2-aminoethyl)-D-myo-inositol-1,4,5-triphosphoric acid (1 mg) in sodium phosphate (100 mM, pH 8.0, 0.5 mL) is added 100 μL of dry acetonitrile. Succinimidyl 6-carboxyfluorescein (1 mg) is dissolved in a minimum of dry DMF (˜100 μL). The activated ester of fluorescein solution is slowly added to the inositol solution, and the reactants mixed by vortex action. The mixture is allowed to stand for 60 minutes to complete the reaction. The product is isolated by HPLC and identified by mass spectroscopy.

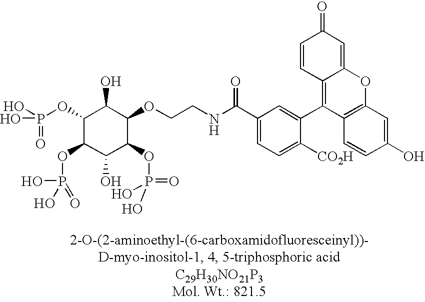

The other fluorescers were conjugated in substantially the same way. To a solution of 2-O-(2′-aminoethyl)-D-myo-inositol-1,4,5-triphosphoric acid triethylammonium salt (0.5 mg) in 0.5 ml of HPLC grade water was added 100 μL of dry acetonitrile. The N-hydroxy succinimide ester of the dye (0.5 mg AlexaFluor 532 and hexachlorofluorescein, Molecular Probes, Eugene, Oreg.; Cy3B, Amersham Biosciences, Buckinghamshire, UK) was dissolved in 100 μl of dry DMF. The dye solution was added to the IP₃ solution while stirring and the reaction was allowed to proceed overnight at room temperature. The product was purified by HPLC on a reverse phase column (C18) with a triethylammonium acetate (100 mM, pH 7.0) water/acetonitrile gradient.

### Example 2

IP3 Binding buffers: Buffer A:50 mM Tris, pH 8.0, 1 mM β-mercaptoethanol, 1 mM EDTA, +1× Complete Protease inhibitor cocktail (from Roche); The IP3 calibrator is resuspended in Buffer A at a stock concentration of 10 mM and then diluted in Buffer A to the various concentrations tested in the assay; The recombinantly expressed IP3 core binding domain protein is diluted in Buffer A (1:150 dilution).

Steps in the assay to generate the calibration curve: Using the EP3 core binding protein, the assay is done in a 384 well white Packard plate. Each reaction that makes up the calibration curve is performed in triplicate. The following is the order of steps of the assay:

1. Pipet 10 μl of IP3 calibrator into the well. The calibrator is titrated from a high concentration of 138 μM to 0.007 μM [final concentration].

2. Add 15 μl of the IP3 binding protein [diluted to a concentration of 0.01 μg/μl] to the well and incubate for 10 minutes at room temperature.

3. Add 10 μl of the ProLabel-IP3 conjugate (0.5 nM reagent concentration) to the well and incubate for 10 minutes at room temperature.

4. Add 10 μl of 0.1× EA to the well and incubate for 30 minutes at room temperature.

5. Add 20 μl of Chemiluminescent substrate (2× reagent concentration) to each of the wells. The plate is incubated at room temperature until ready to read. Read the plate/samples after 15 minutes incubation with the substrate. Additional readings are taken after 30, 60 and 120 minutes. Samples are read on the Packard Lumi-count, with a PMT=1100 and Gain=1.

Analysis of data:

Samples are corrected against background activity in the binding protein samples (ie., RLUs are measured when the binding protein and buffer is incubated with the chemiluminescent substrate). After the background is subtracted from each triplicate sample, the replicates are averaged. The % Inhibition (“Open reading” {ProLabel-IP3+EA+substrate}−“Close reading” {ProLabel-IP3+IP3 binding protein+EA+Substrate} divided by the “Open reading” and times 100) and % Modulation (RLUs of the Calibrator tested -RLUs of the low calibrator divided by the calibrator tested times 100) are calculated and the data is imported into Prism Graphpad to generate a curve and determine the EC₅₀ of the binding reaction.

The signal to noise ratio is determined by the ratio between the highest calibrator divided by the lowest calibrator.

For the fluorescent polarization assays, the following protocol was employed using DTT in the binding protein (“BP”) buffer.

IP3 binding protein (at a concentration of 0.5 μg/μl or 7.1 μM is diluted from 1:100 to 1:400 to provide a range of 72 to 18 nM) is diluted 1:300 in IP₃ binding protein dilution buffer (BP dilution buffer-10 mM HEPES, 88 mM NaCl, 1 mM KCl, 0.1% BGG, 0.02% Tween-20, 25 mM DTT, pH 7.4). A 1M stock of DTT is prepared and then diluted 1:40 into the BP dilution buffer base to make the IP3 BP dilution buffer. 10 μL of calibrator or cells is added to the wells of a 96 well microtiter plate, with 5 μL of ligand (inducing agent) or water and 5 μL of 0.2N perchloric acid. To the above solution is added 10 μL of the IP3-fluorescent derivative in 500 mM TABS, pH 9. After incubating for 5 min, 20 μL of the BP reagent solution is added and the plate read in a fluorescence polarization reader.

When performing the assay with cells, the protocol was modified as follows. CHO-M1 cells were obtained from Euroscreen (Brussels, Belgium) Cells were grown in F12 Media, 10% FBS, 1× Glu, 500 μg/mL G418. Cells were plated at 2×10⁵ per well. Inductions were done using carbachol or acetylcholine (either at 1 mM final concentration) or a titration of the concentration. Inductions were carried out for 20 seconds before the addition of 0.2N PCA. Additional stable cell lines expressing the Histamine 1 receptor or the Vasopressin 3 receptor were also tested. Inductions were done using Histamine (titrating the concentration). The effects on basal levels of IP₃ detected were tested when the cell number was titrated. The basal levels of IP₃ detection increase with the cell number. Three different cells lines expressing different levels of transfected receptor were tested using both the IP₃ Green and Red Tracer (the green tracer is fluorescein, while the red tracer is Alexa (532 nm) fluorescer) assays. The assay is able to detect IP3 in cell lines with varying amounts of IP₃ receptor.

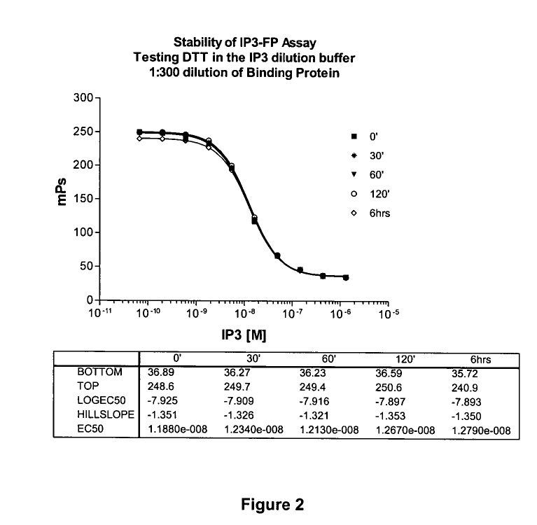

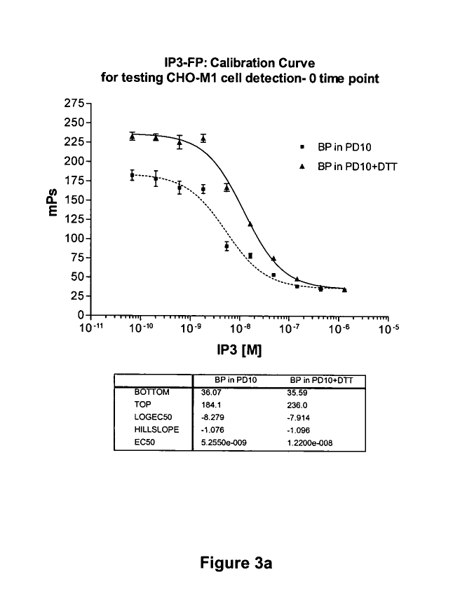

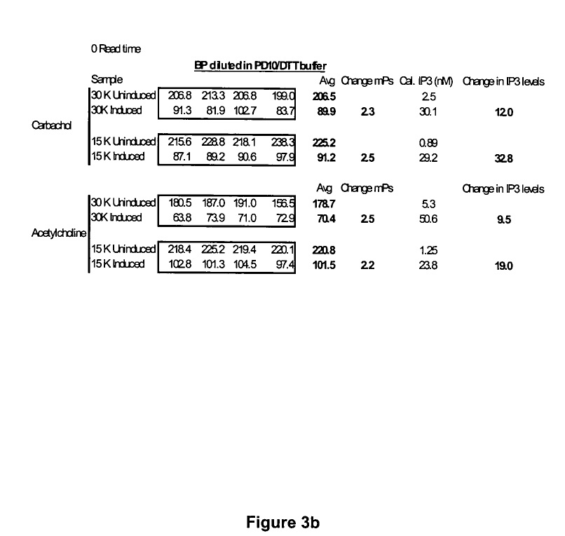

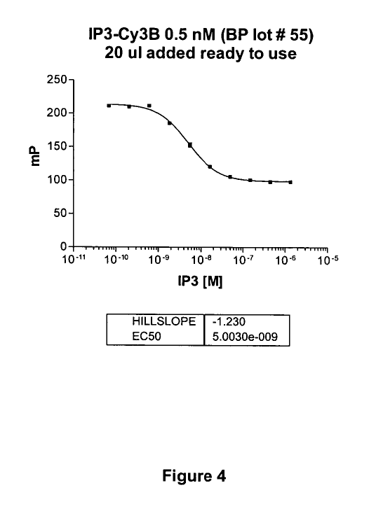

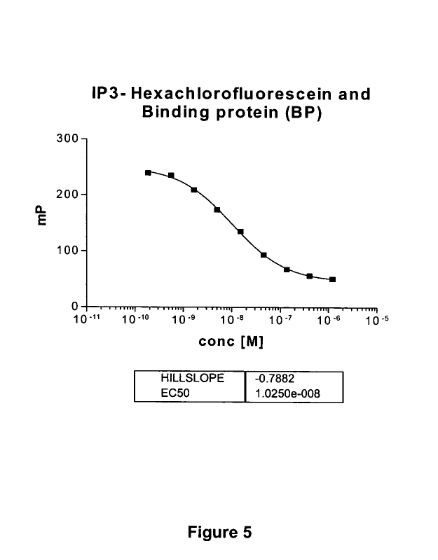

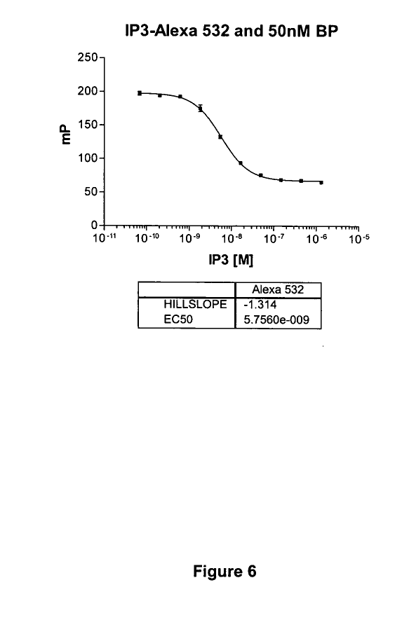

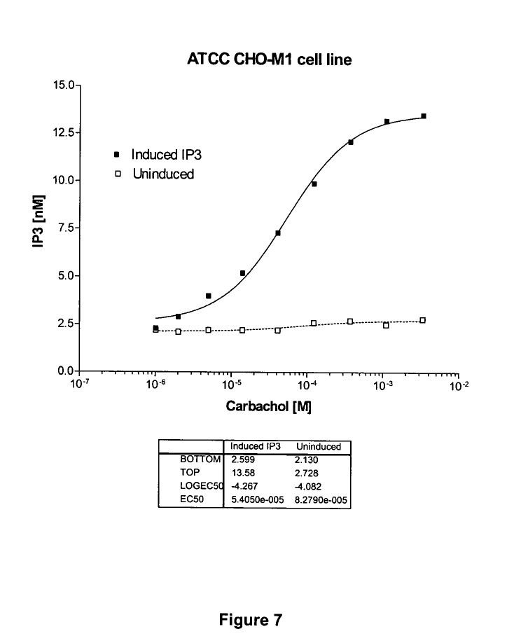

As shown in FIGS. 2-7, using the above protocol, accurate determination of the amount of IP-3 is readily determined over a wide dynamic range with different cell lines, under different conditions of induction.

The subject assays, particularly the homogeneous assays, have many virtues. They are simple to perform and can be readily automated and the results read with currently available equipment. They do not require the tedious separation steps and washings of heterogeneous assays that can introduce technician errors or other errors, even when automated. The assays are specific for IP₃, so that interference from other inositol phosphates is not encountered. The analog of IP₃ competes effectively for the binding protein with IP₃ to provide both sensitivity and specificity.

All references referred to in the text are incorporated herein by reference as if fully set forth herein. The relevant portions associated with this document will be evident to those of skill in the art. Any discrepancies between this application and such reference will be resolved in favor of the view set forth in this application.

Although the invention has been described with reference to the above examples, it will be understood that modifications and variations are encompassed within the spirit and scope of the invention. Accordingly, the invention is limited only by the following claims.

What is claimed is:

1. A protein binding assay for measuring IP₃ in a sample employing as reagents a conjugate of IP₃ and a detectable label joined through a bond or linker at the 2-hydroxyl position, and a truncated extracellular portion of an IP₃R having at least about 200 times the affinity for IP₃ than the intact IP₃R, said method comprising:
combining in an assay medium said sample, said conjugate and said binding protein and incubating said mixture for sufficient time for any IP₃ and said conjugate to bind to said binding protein; and
detecting the bound or unbound label as a measure of the IP₃ present in the sample.

2. A protein binding assay according to claim 1, wherein said assay is in a homogeneous format.

3. A protein binding assay according to claim 1, wherein said sample is a cellular lysate, and wherein said cellular lysate has been treated to block kinases and phosphatases and prepare said sample for said assay.

4. A protein binding assay according to claim 1, wherein said binding protein is of not more than about 600 amino acids and comprises at least amino acids 226-578 of the mouse IP₃R Type 1.

5. A protein binding assay according to claim 1, wherein said label is an enzyme fragment for enzyme complementation.

6. A protein binding assay according to claim 1, wherein said binding protein is a fusion protein of up to about 1.5 kD amino acids.

7. A protein binding assay according to claim 1, wherein said label is a fluorescer.

8. A method according to claim 1, wherein the order of addition of reagents is: (a) combining said sample with said binding protein; and (b) adding said conjugate, with incubating after (a) and (b).

9. A protein binding assay for measuring IP₃ in a sample using a homogeneous format, employing as reagents a conjugate of IP₃ and an ED of from 37 to 60 amino acids derived from β-galactosidase joined through a linker at the 2-hydroxyl position, and a truncated extracellular portion of an IP₃R having at least about 200 times the affinity for IP₃ than the intact IP₃R, said method comprising:
combining in an assay medium assay components in the following order:
said sample, said binding protein, said conjugate and EA, and incubating after each combining for sufficient time for complex formation between said assay components;
adding substrate for said β-galactosidase; and
detecting the turnover of said β-galactosidase of said substrate as a measure of the IP₃ present in the sample.

10. A protein binding assay for measuring IP₃ in a sample using a homogeneous format, employing as reagents a conjugate of IP₃ and a fluorescer joined through a linker at the 2-hydroxyl position, and a truncated extracellular portion of an IP₃R having at least about 200 times the affinity for IP₃ than the intact IP₃R, said method comprising:
combining in an assay medium assay components: said sample, said binding protein, and said conjugate, and incubating for sufficient time for complex formation between said assay components; and
detecting the change in fluorescence polarization as a measure of the IP₃ present in the sample.

11. A method according to claim 10, wherein said linker is an aliphatic group of from 4 to 20 carbon atoms.

12. A method according to claim 9, wherein said fluorescer emits at a wavelength greater than about 500 nm.

13. A method according to claim 10, wherein said fluorescer has a polarizability of less than about 60 mP.

14. A protein binding assay for measuring IP₃ in a sample employing as reagents a conjugate of IP₃ and a detectable label joined through a bond or linker at the 2-hydroxyl position, and a truncated extracellular portion of an IP₃R having at least about 200 times the affinity for IP₃ than the intact IP₃R, said method comprising:
combining in an assay medium said sample, said conjugate, said binding protein and a chemical reductant and incubating said mixture for sufficient time for any IP₃ and said conjugate to bind to said binding protein; and
detecting the bound or unbound label as a measure of the IP₃ present in the sample.

15. A protein binding assay according to claim 14, wherein said chemical reductant is a thiol.

16. A compound of the formula: 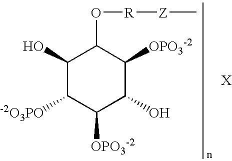
wherein:
R is a neutral linking group of from 4 to 20 carbon atoms bonded to the oxygen through a saturated carbon atom or carbonyl;
Z is a functionality for linking X to the oxygen at the 2-position;
X is an enzyme donor fragment of β-galactosidase of from 27 to 60 amino acids; and
n is 1 or 2.

17. A compound of the formula: 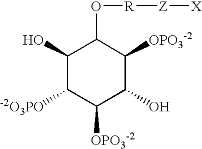
wherein:
R is a neutral linking group of from 2 to 20 carbon atoms bonded to the oxygen through a saturated carbon atom;
Z is a functionality for linking X to the oxygen at the 2-position; and
X is a fluorescer.

18. A kit comprising a compound according to claim 17, enzyme acceptor for said enzyme donor and a truncated extracellular portion of an IP₃R having at least about 200 times the affinity for IP₃ than the intact IP₃R.

19. A kit comprising a compound according to claim 18, enzyme acceptor for said enzyme donor and a truncated extracellular portion of an IP₃R having at least about 200 times the affinity for IP₃ than the intact IP₃R.

20. A kit for performing an IP3 assay comprising a conjugate of IP₃ and a detectable label joined through a bond or linker at the 2-hydroxyl position, a truncated extracellular portion of an IP₃R having at least about 200 times the affinity for IP₃ than the intact IP₃R and instructions for performing said assay.

21. A kit according to claim 20, further comprising a thiol reductant.
